(19) United States
(12) Patent Application Publication
(10) Pub. No.: US 2023/0415178 A1
(43) Pub. Date: Dec. 28, 2023

(54) ATOMIZER

(71) Applicant: Murata Manufacturing Co., Ltd., Kyoto (JP)

(72) Inventors: Miki IKEDA, Kyoto (JP); Kenghua HSING, Kyoto (JP)

(21) Appl. No.: 18/463,611

(22) Filed: Sep. 8, 2023

Related U.S. Application Data
(63) Continuation of application No. PCT/JP2022/003393, filed on Jan. 28, 2022.

(30) Foreign Application Priority Data
Mar. 26, 2021 (JP) 2021-053575

(51) Int. Cl. B05B 7/04; A61M 11/02

(52) U.S. Cl. CPC B05B 7/0425; A61M 11/02

(57) ABSTRACT

An atomizer includes a gas supply member provided with a gas supply port configured to supply a gas and a liquid supply member provided with a liquid supply port configured to supply a liquid. The gas supply member has a gas supply surface as a surface to form the gas supply port, the liquid supply port is opened toward an axis orthogonal to the gas supply surface at the gas supply port, and the liquid supply member has a first inclined surface between the liquid supply port and the gas supply port. The first inclined surface is inclined to leave from an axis orthogonal to the gas supply port as the first inclined surface extends away from the gas supply surface in a first section including a gas flow path and a liquid flow path.

### CROSS REFERENCE TO RELATED APPLICATION

This is a continuation of International Application No. PCT/JP2022/003393 filed on Jan. 28, 2022 which claims priority from Japanese Patent Application No. 2021-053575 filed on Mar. 26, 2021. The contents of these applications are incorporated herein by reference in their entireties.

### BACKGROUND OF THE DISCLOSURE

### Field of the Disclosure

The present disclosure relates to an atomizer for mixing a liquid and a gas to atomize.

### Description of the Related Art

An atomizer for mixing a liquid and a gas to atomize has been disclosed in the past (see Patent Document 1, for example).

The atomizer of Patent Document 1 atomizes using the Venturi effect. Specifically, a liquid stored in a reservoir is sucked out by blowing the compressed air out from a nozzle hole to generate the negative pressure around the nozzle hole, and the sucked liquid is mixed with the compressed air to atomize.
Patent Document 1: Japanese Unexamined Patent Application Publication No. 2013-132471

### BRIEF SUMMARY OF THE DISCLOSURE

An atomization amount needs to be increased in a configuration including the configuration disclosed in Patent Document 1.

Accordingly, a possible benefit of the present disclosure is to solve the above-mentioned issue and to provide an atomizer capable of increasing the atomization amount.

In order to achieve the possible benefit above, the atomizer of the present disclosure is an atomizer for mixing a gas and a liquid to atomize, and includes a gas supply member provided with a gas flow path and a gas supply port configured to supply a gas and a liquid supply member provided with a liquid flow path and a liquid supply port configured to supply a liquid. The gas supply member has a gas supply surface as a surface to form the gas supply port, the liquid supply port is opened toward an axis orthogonal to the gas supply surface at the gas supply port, and the liquid supply member has a first inclined surface between the liquid supply port and the gas supply port. The first inclined surface is inclined to leave from the axis as the first inclined surface extends away from the gas supply surface in a first section including the gas flow path and the liquid flow path. The liquid supply port is located at a position protruding from a plane including the first inclined surface.

With the use of the atomizer of the present disclosure, the atomization amount may be increased.

### BRIEF DESCRIPTION OF THE SEVERAL VIEWS OF THE DRAWINGS

FIG. 1 is a perspective view of an atomizer according to Embodiment 1.

FIG. 2 is a perspective view of the atomizer according to Embodiment 1.

FIG. 3 is a top view of the atomizer according to Embodiment 1.

FIG. 4 is a bottom view of the atomizer according to Embodiment 1.

FIG. 5 is a perspective view of the atomizer according to Embodiment 1 in a state with a third case removed.

FIG. 6 is a perspective view of the atomizer according to Embodiment 1 in a state with the third case removed.

FIG. 7 is a perspective view of a support member according to Embodiment 1.

FIG. 8 is a perspective view of the atomizer with the support member further omitted from the atomizer illustrated in FIG. 5 and FIG. 6.

FIG. 9 is a perspective view of the atomizer according to Embodiment 1 illustrating a longitudinal section thereof.

FIG. 10A is a perspective view of a first case according to Embodiment 1 illustrating a longitudinal section thereof.

FIG. 10B is a perspective view of the first case according to Embodiment 1 illustrating a longitudinal section thereof.

FIG. 11A is a perspective view of a liquid supply member according to Embodiment 1 illustrating a longitudinal section thereof.

FIG. 11B is a perspective view of the liquid supply member according to Embodiment 1 illustrating an entire body thereof.

FIG. 12A is a perspective view of an atomization portion in Embodiment 1.

FIG. 12B is a perspective view of the atomization portion in Embodiment 1 illustrating a longitudinal section.

FIG. 13 is a longitudinal sectional view of the atomization portion according to Embodiment 1 illustrating a peripheral configuration thereof.

FIG. 14 is an enlarged perspective view of the atomization portion according to Embodiment 1.

FIG. 15 is an enlarged longitudinal sectional view of the atomization portion according to Embodiment 1.

FIG. 16 is an enlarged plan view of the atomization portion according to Embodiment 1.

FIG. 17 is a plan view of a gas supply port according to Embodiment 1.

FIG. 18 is a plan view of a liquid supply port according to Embodiment 1.

FIG. 19A is a longitudinal sectional view of an atomization portion including a liquid supply member according to Modification 1.

FIG. 19B is a longitudinal sectional view of an atomization portion including a liquid supply member according to Modification 2.

FIG. 19C is a longitudinal sectional view of an atomization portion including a liquid supply member according to Modification 3.

FIG. 19D is a longitudinal sectional view of an atomization portion including a liquid supply member according to Modification 4.

FIG. 19E is a longitudinal sectional view of an atomization portion including a liquid supply member according to Modification 5.

FIG. 19F is a longitudinal sectional view of an atomization portion including a liquid supply member according to Modification 6.

FIG. 19G is a longitudinal sectional view of an atomization portion including a liquid supply member according to Modification 7.

FIG. 19H is a longitudinal sectional view of an atomization portion including a liquid supply member according to Modification 8.

FIG. 20 is a perspective view of an atomizer according to Embodiment 2.

### DETAILED DESCRIPTION OF THE DISCLOSURE

According to a first aspect of the present disclosure, the atomizer is an atomizer for mixing a gas and a liquid to atomize, and includes a gas supply member provided with a gas flow path and a gas supply port configured to supply a gas and a liquid supply member provided with a liquid flow path and a liquid supply port configured to supply a liquid. The gas supply member has a gas supply surface as a surface to form the gas supply port, the liquid supply port is opened toward an axis orthogonal to the gas supply surface at the gas supply port, and the liquid supply member has a first inclined surface between the liquid supply port and the gas supply port. The first inclined surface is inclined to leave from the axis as the first inclined surface extends away from the gas supply surface in a first section including the gas flow path and the liquid flow path. The liquid supply port is located at a position protruding from a plane including the first inclined surface.

According to a second aspect of the present disclosure, there is provided the atomizer according to the first aspect which satisfy the following. The liquid supply member has a second inclined surface on an upstream side relative to the first inclined surface in a gas flow direction and at a position facing a gas blown out from the gas supply port, and in the first section, the second inclined surface is inclined to approach the axis as the second inclined surface extends away from the gas supply surface.

According to a third aspect of the present disclosure, there is provided the atomizer according to the second aspect, in which the first inclined surface and the second inclined surface are connected by a ridge line.

According to a fourth aspect of the present disclosure, there is provided the atomizer according to the third aspect, in which the ridge line has a shape approaching an upstream side of the liquid supply port in a liquid flow direction as the ridge line leaves from the gas supply port in a plan view direction of the gas supply port.

According to a fifth aspect of the present disclosure, there is provided the atomizer according to any one of the first to fourth aspects, in which the liquid supply member further has a liquid supply surface to form the liquid supply port.

According to a sixth aspect of the present disclosure, there is provided the atomizer according to the fifth aspect, in which the liquid supply surface extends substantially parallel to the axis.

According to a seventh aspect of the present disclosure, there is provided the atomizer according to any one of the first to sixth aspects, in which the maximum measurement of an opening of the liquid supply port in a lateral direction orthogonal to the first section is larger than the maximum measurement of the opening of the liquid supply port in a longitudinal direction intersecting with the lateral direction.

According to an eighth aspect of the present disclosure, there is provided the atomizer according to any one of the first to seventh aspects, in which the maximum measurement of the gas supply port in a lateral direction orthogonal to the first section is larger than the maximum measurement of the opening of the gas supply port in a longitudinal direction intersecting with the lateral direction.

According to a ninth aspect of the present disclosure, there is provided the atomizer according to any one of the first to eighth aspects, further including a piezoelectric pump configured to supply a gas to the gas supply port.

According to a tenth aspect of the present disclosure, there is provided the atomizer according to any one of the first to ninth aspects, in which the gas supply member and the liquid supply member are separate members.

Hereinafter, embodiments according to the present disclosure will be described in detail with reference to the drawings.

### Embodiment 1

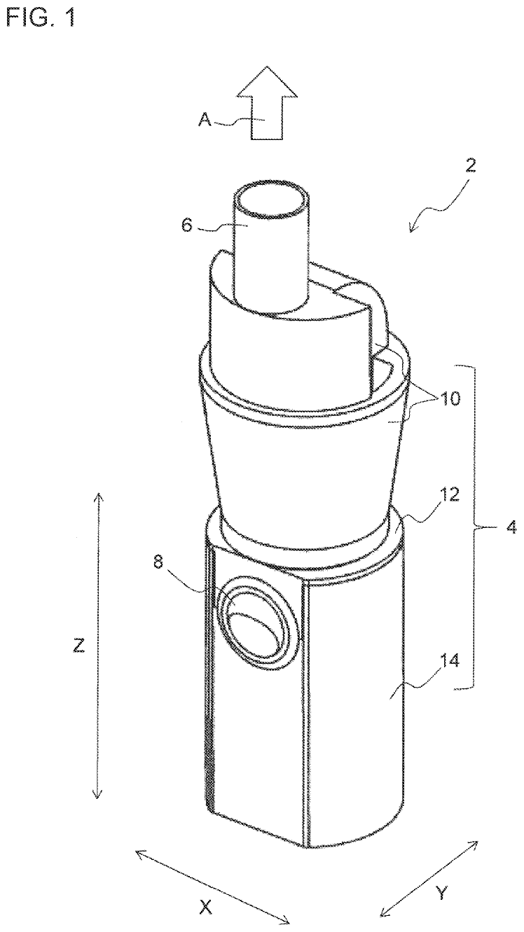

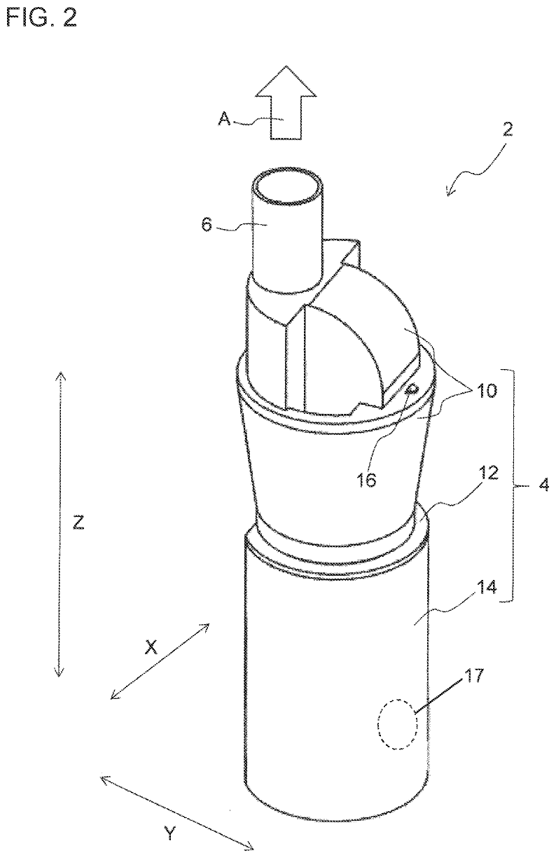

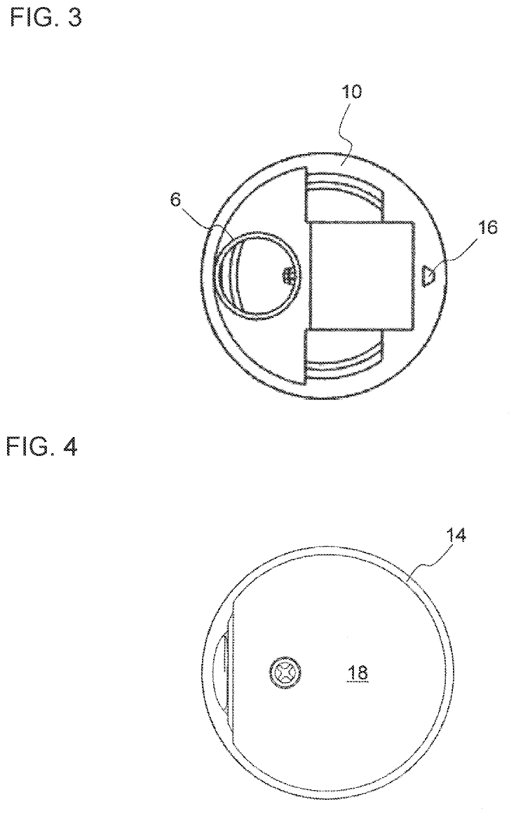

FIG. 1 to FIG. 4 are drawings each illustrating an atomizer 2 according to Embodiment 1 of the present disclosure. FIG. 1 and FIG. 2 each are a perspective view of the atomizer 2, FIG. 3 is a top view of the atomizer 2, and FIG. 4 is a bottom view of the atomizer 2.

The atomizer 2 is a device for mixing a liquid and a gas to atomize. The atomizer 2 is used as a medical nebulizer, for example. The liquid is saline, an organic solvent (ethanol or the like), a drug (steroid, (2 stimulant or the like), for example, and the gas is air, for example.

The atomizer 2 illustrated in FIG. 1 and FIG. 2 includes a case 4, a blowout nozzle 6, and a switch 8. The atomizer 2 of Embodiment 1 is a handheld atomizer that can be used alone without being connected to another device. A driving battery (not illustrated) may be built in the atomizer 2. When a user presses the switch 8, an atomized liquid is blown out from the blowout nozzle 6 (see arrow A). As illustrated in FIG. 1 and FIG. 2, with respect to the direction of the atomizer 2 in a state standing by itself, a left-right direction is an X-direction, a front-rear direction is a Y-direction, an up-down direction is a Z-direction. The X-direction and the Y-direction each are also referred to as a “lateral direction”.

The case 4 is a member to house the internal components of the atomizer 2 and constitutes an outer frame of the atomizer 2. The case 4 includes a first case 10, a second case 12, and a third case 14. The first case 10 of an upper stage and the second case 12 of a middle stage are engaged with each other, and the second case 12 of the middle stage and the third case 14 of a lower stage are engaged with each other.

The blowout nozzle 6 is a nozzle formed to protrude from the first case 10. The blowout nozzle 6 protrudes upward from an upper surface of the atomizer 2 and forms a flow path and an opening through which the atomized liquid is blown out.

The switch 8 is a member for switching ON and OFF of the operation of the atomizer 2. The switch 8 is provided on a front surface side of the atomizer 2 in the same manner as the blowout nozzle 6, and is disposed between the second case 12 and the third case 14.

As illustrated in FIG. 2 and FIG. 3, a mark 16 is provided on an upper surface of the first case 10. The mark 16 allows a user to easily recognize the orientation of the atomizer 2. The mark 16 of Embodiment 1 is a triangular arrow in plan view.

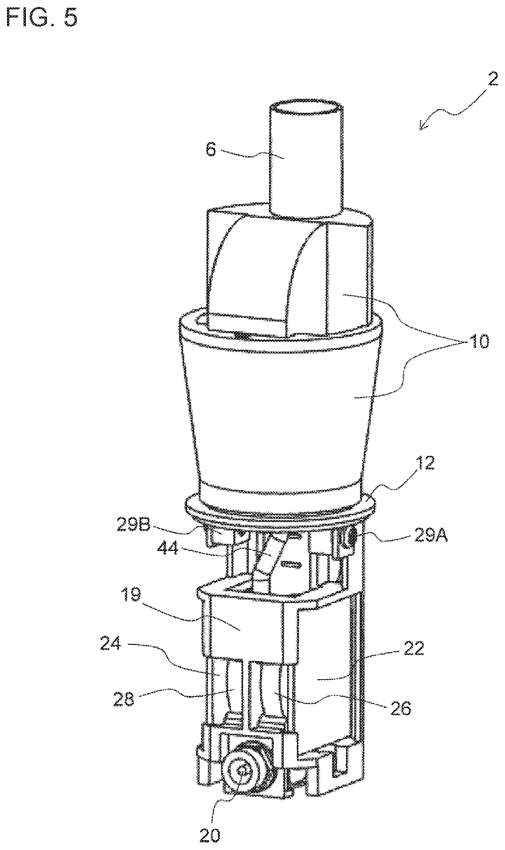

As illustrated in FIG. 2, the third case 14 may be provided with a power supply cover 17. The power supply cover 17 is detachably provided at a position to cover a power supply plug portion 20 (FIG. 5) described later. The power supply cover 17 is not necessarily provided since a simple opening may be provided instead thereof, for example. Being provided with the power supply cover 17 is more preferable since the power supply portion can be sealed.

As illustrated in FIG. 4, the third case 14 has a bottom surface 18. The bottom surface 18 constitutes a bottom surface of the atomizer 2, and has a flat shape so that the atomizer 2 can stand by itself.

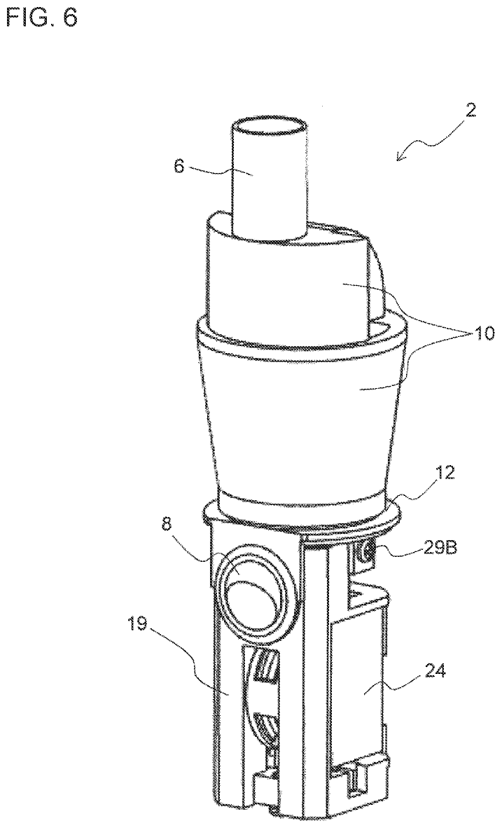

FIG. 5 and FIG. 6 each are a perspective view of the atomizer 2 from which the third case 14 is removed.

As illustrated in FIG. 5 and FIG. 6, a support member 19, the power supply plug portion 20, two control boards 22 and 24, and two piezoelectric pumps 26 and 28 are provided inside the atomizer 2.

The support member 19 supports members such as the power supply plug portion 20, the control boards 22 and 24, the piezoelectric pumps 26 and 28, and the switch 8 (FIG. 6). The support member 19 is fixed to the second case 12 by screws 29A and 29B. The power supply plug portion 20 is a member in which an opening for plugging a power supply such as an AC power supply is formed. The power supply plug portion 20 is electrically connected to each of the control boards 22 and 24 by a wiring line (not illustrated). Power can be supplied to the control boards 22 and 24 and the piezoelectric pumps 26 and 28 by plugging a power supply into the power supply plug portion 20. The control boards 22 and 24 are circuit boards for driving the piezoelectric pumps 26 and 28, respectively. The control board 22 applies a drive voltage to drive the piezoelectric pump 26 at a predetermined frequency (20 kHz, for example), and the control board 24 applies a drive voltage to drive the piezoelectric pump 28 at a predetermined frequency (20 kHz, for example).

Each of the piezoelectric pumps 26 and 28 is a piezoelectric pump using a piezoelectric element (may be referred to as a “microblower”, a “micropump”, or the like). Specifically, it has a structure in which a piezoelectric element (not illustrated) is bonded to a metal plate (not illustrated), and by supplying AC power to the piezoelectric element and the metal plate, bending deformation of a unimorph mode is generated to transport a gas. Such a piezoelectric pump has a built-in diaphragm (not illustrated) having a valve function for restricting the flow of a gas in one direction.

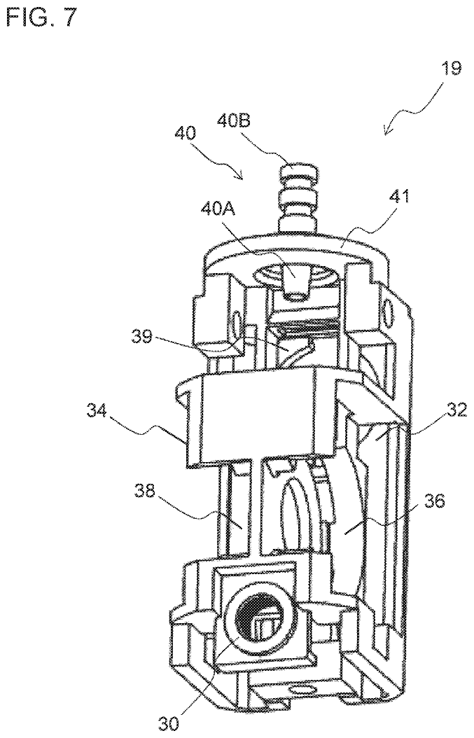

A perspective view of the support member 19 is illustrated in FIG. 7. As illustrated in FIG. 7, the support member 19 has a plurality of mount portions, that is, mount portions 30, 32, 34, 36, 38, and 39. The mount portion 30 is an opening portion for mounting the power supply plug portion 20, and is provided on a rear surface side of the support member 19. The mount portions 32 and 34 are opening portions for mounting the control boards 22 and 24, respectively. The mount portions 36 and 38 are opening portions for mounting the piezoelectric pumps 26 and 28, respectively. The mount portion 39 is an opening portion for mounting the switch 8, and is provided on a front surface side of the support member 19.

The support member 19 further includes a nozzle portion 40. The nozzle portion 40 is a cylindrical member in which a flow path, for flowing the air generated by the piezoelectric pumps 26 and 28 to a downstream side, is formed. The nozzle portion 40 is formed to penetrate through an upper surface portion 41 of the support member 19, and has an upstream end 40A on one side (that is, lower side) and a downstream end 40B on the other side (that is, upper side) relative to the upper surface portion 41.

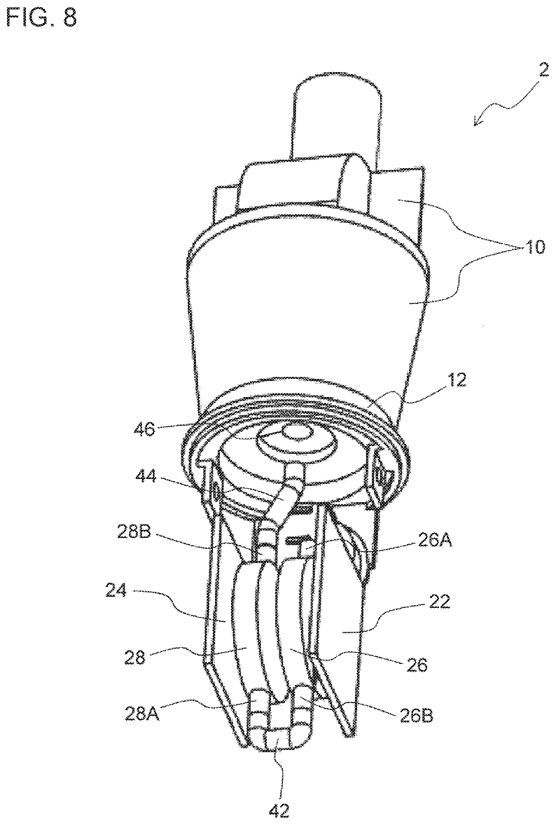

FIG. 8 is a perspective view of the atomizer 2 with the support member 19 further omitted from the atomizer 2 illustrated in FIG. 5 and FIG. 6.

As illustrated in FIG. 8, two connection flow path members 42 and 44 are further provided inside the atomizer 2. The connection flow path member 42 is a cylindrical member that forms a flow path for connecting the piezoelectric pumps 26 and 28 to each other. The connection flow path member 44 is a cylindrical member that forms a flow path for connecting the piezoelectric pump 28 to the downstream side. The piezoelectric pumps 26 and 28 are connected in series by the connection flow path member 42. By providing the two piezoelectric pumps 26 and 28 as a gas supply source, the supply amount of a gas may be increased.

The piezoelectric pump 26 has an upstream end 26A and a downstream end 26B. The upstream end 26A is open to the atmosphere, and the downstream end 26B is connected to the connection flow path member 42. The piezoelectric pump 28 has an upstream end 28A and a downstream end 28B. The upstream end 28A is connected to the connection flow path member 42, and the downstream end 28B is connected to the connection flow path member 44.

According to a flow path configuration illustrated in FIG. 8, the piezoelectric pump 26 sucks air from the upstream end 26A and discharges the air to the connection flow path member 42 via the downstream end 26B. The piezoelectric pump 28 sucks the air supplied from the connection flow path member 42 from the upstream end 28A, and discharges the air to the connection flow path member 44 via the downstream end 28B.

The connection flow path member 44 is connected to the upstream end 40A of the nozzle portion 40 illustrated in FIG. 7. An upper portion of the nozzle portion 40, which includes the downstream end 40B and protrudes from the upper surface portion 41, is inserted into an opening 46 provided in a bottom portion of the first case 10 illustrated in FIG. 8.

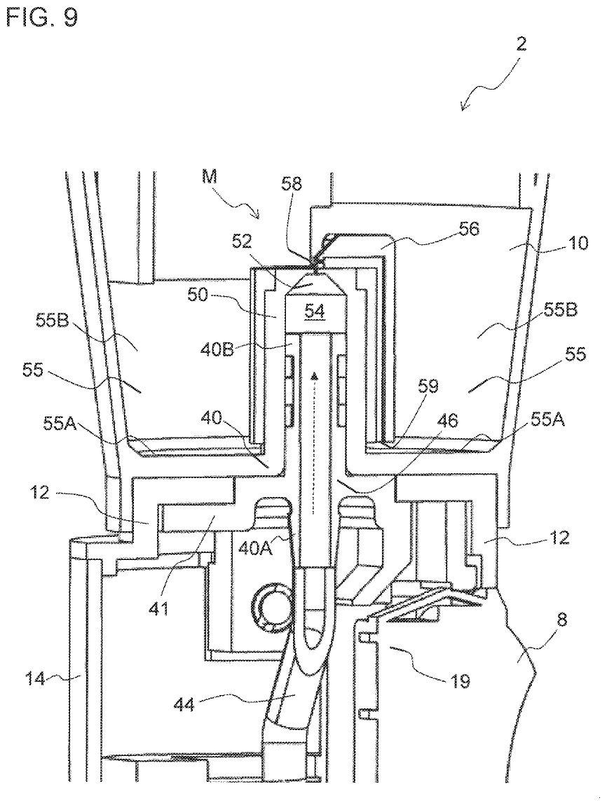

A peripheral configuration of the opening 46 of the first case 10 will be described with reference to FIG. 9 to FIG. 13.

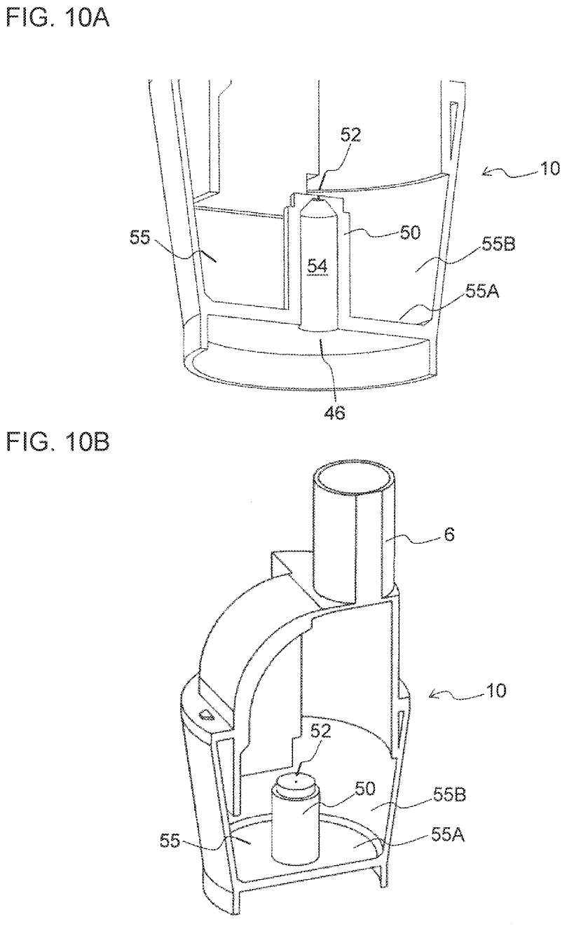

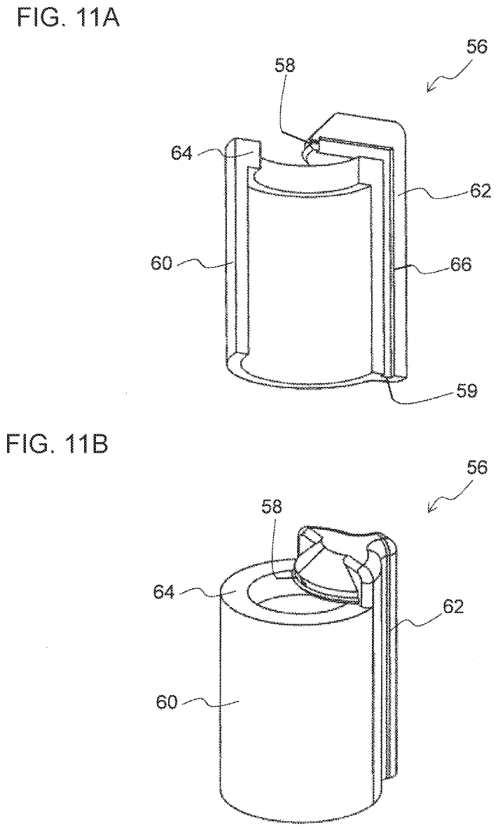

FIG. 9 is a perspective view of the atomizer 2 illustrating a longitudinal section thereof. FIG. 10A and FIG. 10B each are a perspective view of the first case 10 illustrating a longitudinal section, and FIG. 11A and FIG. 11B respectively are a perspective view of a liquid supply member 56 illustrating a longitudinal section thereof and a perspective view of the liquid supply member 56 illustrating an entire body thereof.

As illustrated in FIG. 9, the nozzle portion 40 is inserted into a gas supply member 50 provided in the first case 10 via the opening 46 of the first case 10. The gas supply member 50 is a cylindrical portion having a gas supply port 52 formed at a tip thereof, and has a gas flow path 54 formed therein. The gas supply member 50 of Embodiment 1 is provided integrally with the first case 10 and is positioned at a center of the first case 10. The first case 10 including the gas supply member 50 may be referred to as the “gas supply member”.

The gas flow path 54 extends from the opening 46 of the first case 10 to the gas supply port 52. The gas flow path 54 allows a gas supplied from the downstream end 40B of the nozzle portion 40 inserted into the opening 46 to flow to the gas supply port 52. As illustrated in FIG. 9, in a state that the nozzle portion 40 is inserted into the gas supply member 50, a gas supplied from the nozzle portion 40 is blown out upward from the gas supply port 52 via the gas flow path 54 of the gas supply member 50.

The liquid supply member 56 is mounted on the outside of the gas supply member 50. The liquid supply member 56 forms a liquid supply port 58 for supplying a liquid. The liquid supply member 56 further forms a liquid suction port 59 at a bottom thereof to suck in a liquid. The liquid supply member 56 of Embodiment 1 is a member separate from the gas supply member 50.

As illustrated in FIG. 9, a liquid reservoir 55 is provided around the gas supply member 50 and the liquid supply member 56. The liquid reservoir 55 stores a liquid to be supplied to the liquid supply member 56. The liquid reservoir 55 of Embodiment 1 is formed by a bottom surface provided inside the first case 10 and an inner peripheral surface 55B adjacent to the bottom surface 55A. The liquid reservoir 55 faces the liquid suction port 59 of the liquid supply member 56. In the drawing, a liquid stored in the liquid reservoir 55 is not illustrated.

As illustrated in FIG. 11A and FIG. 11B, the liquid supply member 56 includes a mount portion 60 and a flow path forming portion 62.

The mount portion 60 is a portion at which the liquid supply member 56 is attached to the gas supply member described above. The mount portion 60 is formed in a cylindrical shape, and has a shape in which an upper end portion 64 protrudes inward. The gas supply member 50 is disposed in an internal space of the mount portion 60, and the liquid supply member 56 is attached to the outside of the gas supply member 50 in a state that an upper surface of the gas supply member 50 is in contact with the upper end portion 64 of the mount portion 60.

The flow path forming portion 62 forms a liquid flow path 66. The liquid flow path 66 extends from the liquid supply port 58 to the liquid suction port 59. The liquid flow path 66 of Embodiment 1 extends upward from the liquid suction port 59, bends at a substantially right angle, and extends in a lateral direction to the liquid supply port 58.

As illustrated in FIG. 9, in a state that the liquid supply member 56 is attached to the outer side of the gas supply member 50, the gas supply port 52 and the liquid supply port 58 are disposed at positions close to each other. The gas supply member 50 to form the gas supply port 52 and the liquid supply member 56 to form the liquid supply port 58 constitute an “atomization portion M” that mixes a gas and a liquid to atomize.

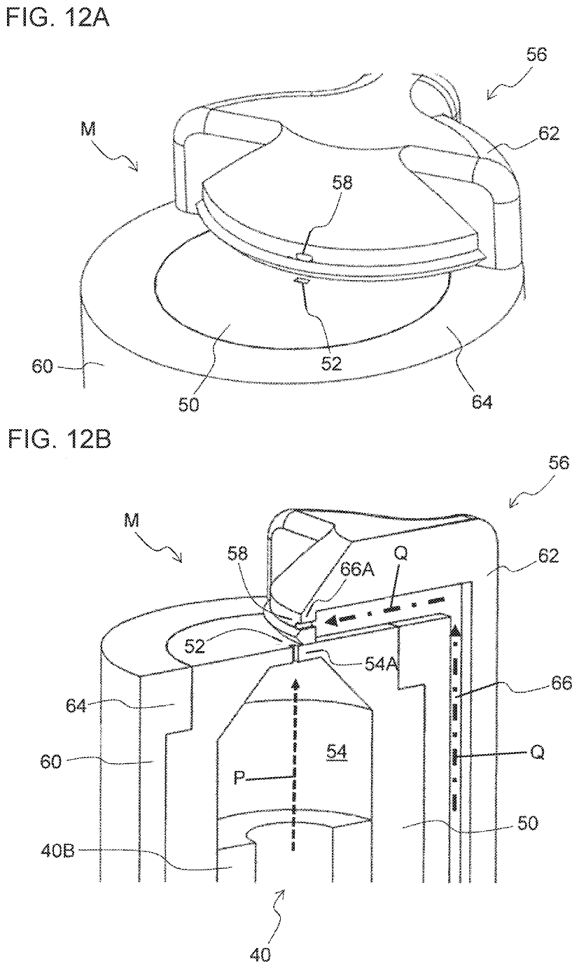

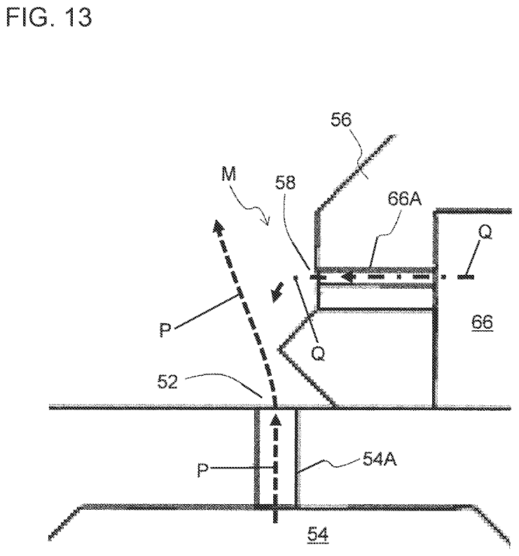

The peripheral configuration of the atomization portion M will be described with reference to FIG. 12A, FIG. 12B and FIG. 13. FIG. 12A is a perspective view of the atomization portion M illustrating a peripheral configuration thereof, and FIG. 12B is a perspective view of the atomization portion M illustrating a longitudinal section including the peripheral configuration thereof. FIG. 13 is a longitudinal sectional view of the atomization portion M illustrating a peripheral configuration thereof.

As illustrated in FIG. 12A, FIG. 12B, and FIG. 13, in a state that the gas supply port 52 and the liquid supply port 58 are close to each other, the gas supply port 52 opens upward, and the liquid supply port 58 opens in a lateral direction (rearward of atomizer 2). As illustrated in FIG. 13, the liquid supply port 58 opens in a direction facing a flow P of a gas blown out from the gas supply port 52.

As illustrated in FIG. 13 and FIG. 12B, the gas supply port 52 is positioned at a tip of a shrunk diameter portion 54A in which a diameter of the gas flow path 54 is made small. Providing the shrunk diameter portion 54A makes only the vicinity of the output port of the gas flow path 54 narrow, and this allows air to be carried with low resistance to the vicinity of the gas supply port 52 in the gas flow path 54. Thus, a flow velocity of the air blown out from the gas supply port 52 may be increased. Similarly, the liquid supply port 58 is positioned at a tip of a shrunk diameter portion 66A in which a diameter of the liquid flow path 66 is made small. With the use of the flow path configuration described above, the atomization with the Venturi effect may be performed in accordance with the flow P of the gas blown out from the gas supply port 52.

Here, the operation of the atomizer 2 to perform the atomization with the Venturi effect will be described. First, a user presses the switch 8 to activate the atomizer 2. Control boards 22 and 24 respectively drive piezoelectric pumps 26 and 28 to generate the compressed air. A gas as the compressed air generated by the piezoelectric pumps 26 and 28 is blown upward from the gas supply port 52 via the nozzle portion 40.

Negative pressure is generated in a peripheral region including the liquid supply port 58 in accordance with the flow P of the gas from the gas supply port 52. With this, a liquid stored in the liquid reservoir 55 is sucked into the liquid flow path 66 from the liquid suction port 59, and a flow Q of the liquid flowing toward the liquid supply port 58 is generated (Venturi effect). The flow Q of the liquid discharged from the liquid supply port 58 to the outside is atomized by being mixed with the flow P of the gas being the compressed air. The atomized liquid moves upward in an internal space of the first case 10, and is blown out from the blowout nozzle 6 while being classified.

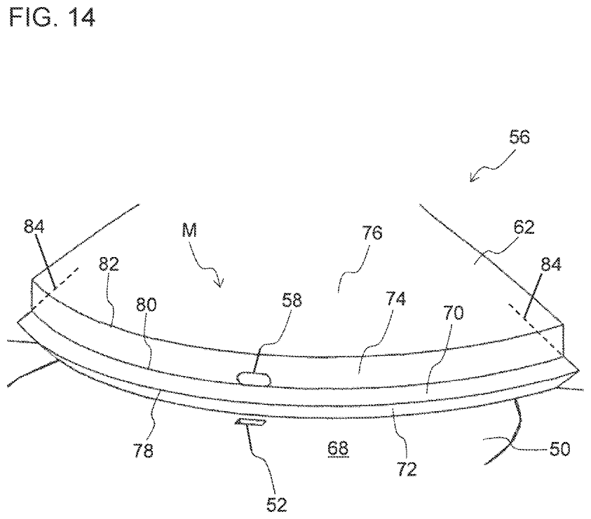

In the atomizer 2 having the configuration described above, the shape and the like of the liquid supply member 56 are devised in order to increase an atomization amount of the atomization portion M. Specifically, a description will be made with reference to FIG. 14 to FIG. 16.

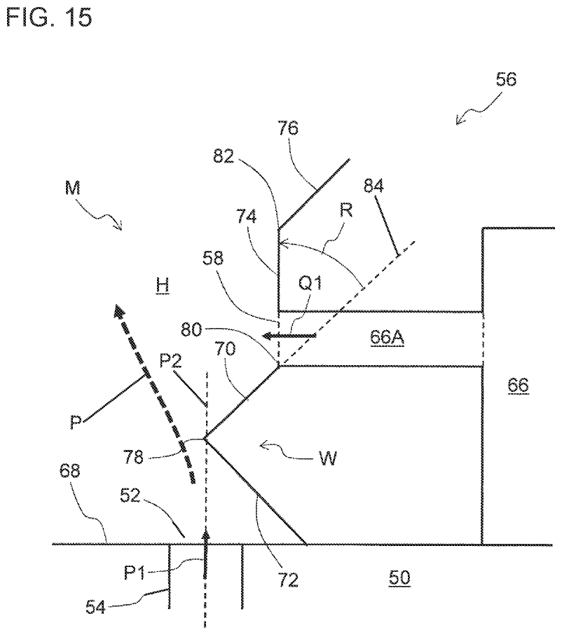

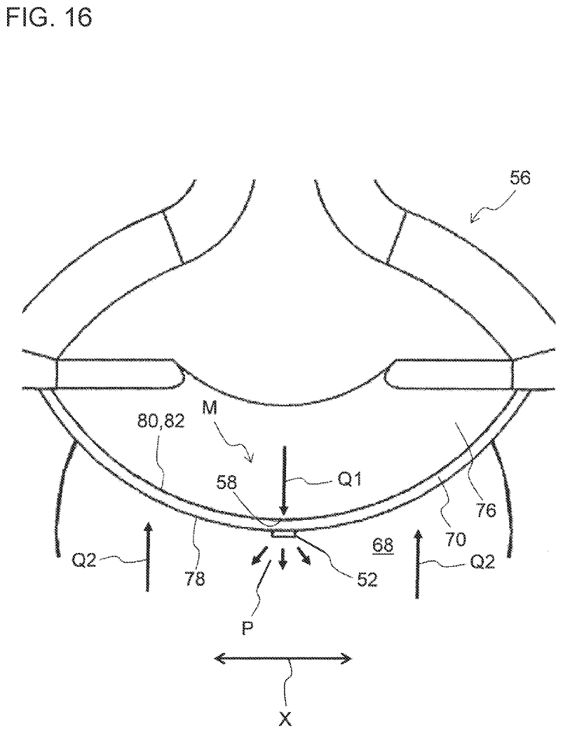

FIG. 14 is an enlarged perspective view of the atomization portion M, FIG. 15 is an enlarged longitudinal sectional view of the atomization portion M, and FIG. 16 is an enlarged plan view of the atomization portion M. FIG. 15, in particular, illustrates a section (first section) including a gas flow direction P1 at the gas supply port 52 and a liquid flow direction Q1 at the liquid supply port 58. In other words, FIG. 15 is a section including the gas flow path 54 and the liquid flow path 66. FIG. 16 is a drawing in a plan view direction of the gas supply port 52.

As illustrated in FIG. 14 and FIG. 15, the gas supply member 50 has a gas supply surface 68 as a surface to form the gas supply port 52. The gas supply surface 68 of Embodiment 1 has a flat shape, and the gas supply port 52 is formed to be flush with the gas supply surface 68.

As illustrated in FIG. 15, the gas flow direction P1 at the shrunk diameter portion 54A can be defined as a direction in which the shrunk diameter portion 54A extends at the gas supply port 52. Since the gas flow path 54 of Embodiment 1 is substantially perpendicularly connected to the gas supply surface 68, the gas flow direction P1 at the gas supply port 52 is substantially perpendicular to the gas supply surface 68.

The liquid supply member 56 has a first inclined surface 70, a second inclined surface 72, a liquid supply surface 74, and a third inclined surface 76. The second inclined surface 72, the first inclined surface 70, the liquid supply surface 74, and the third inclined surface 76 are provided in this order from the upstream side in the gas flow direction P1.

As illustrated in FIG. 15, the first inclined surface 70, the second inclined surface 72, and the third inclined surface 76 are all inclined relative to the gas flow direction P1 at the gas supply port 52 and an axis P2 including the gas flow direction P1. The axis P2 is an imaginary straight line orthogonal to the gas supply port 52, and when a minimum circle including the gas supply port 52 is drawn, the axis P2 is an imaginary line at a center of the circle. In other words, the axis P2 is a virtual straight line orthogonal to the gas supply surface 68 at the gas supply port 52. The first inclined surface 70 and the third inclined surface 76, in particular, are inclined in a direction leaving from the axis P2 including the gas flow direction P1 as the inclined surfaces extend away from the gas supply surface 68 along the gas flow direction P1. On the other hand, the second inclined surface 72 is inclined in a direction approaching the axis P2 including the gas flow direction P1 as the second inclined surface 72 extends away from the gas supply surface 68 along the gas flow direction P1.

The liquid supply surface 74 is a surface to form the liquid supply port 58. The liquid supply surface 74 is formed between the first inclined surface 70 and the third inclined surface 76, and connects the first inclined surface 70 and the third inclined surface 76. The liquid supply surface 74 of Embodiment 1 is a surface substantially parallel to the gas flow direction P1 and the axis P2 at the gas supply port 52. The liquid supply surface 74 of Embodiment 1 forms the liquid supply port 58 in a lower end portion thereof. As a result, the liquid supply port 58 is formed to be continuous with the first inclined surface 70 and a ridge line 80 described later.

In Embodiment 1, the first inclined surface 70 and the second inclined surface 72 are continuously formed and are connected to each other by a ridge line 78. Similarly, the first inclined surface 70 and the liquid supply surface 74 are continuously formed and are connected to each other by the ridge line 80, and the liquid supply surface 74 and the third inclined surface 76 are continuously formed and are connected to each other by a ridge line 82.

As illustrated in FIG. 15 and FIG. 14, the second inclined surface 72 is disposed with an angle relative to the flow direction P1 of the gas blown out from the gas supply port 52 and the axis P2. The gas blown out from the gas supply port 52 collides with the second inclined surface 72 and is blown out upward while being curved in a direction leaving from the liquid supply member 56. On the other hand, the first inclined surface 70 is inclined in a direction opposite to that of the second inclined surface 72. With this, a peripheral region of the first inclined surface 70 is recessed relative to the gas flow P, and the negative pressure is not likely to diffuse to the periphery. Thus, the negative pressure increases. Since the liquid supply port 58 is provided in the vicinity of the first inclined surface 70 being a negative pressure generation region, a strong negative pressure is generated around the liquid supply port 58. This makes it possible to suck the liquid with a strong suction force.

Particularly in Embodiment 1, in the section illustrated in FIG. 15, the liquid supply port 58 is provided at a position protruding relative to a virtual plane 84 including the first inclined surface 70 (see arrow R). The virtual plane 84 is flush with the first inclined surface 70. With the disposition of the liquid supply port 58 described above, the liquid supply port 58 may be disposed at a place close to the gas flow P in comparison with a case being provided at a position flush with the virtual plane 84 including the first inclined surface 70, that is, the place at which strong negative pressure is generated. With this, a suction force of a liquid due to the Venturi effect may be increased, and the atomization amount may be increased.

By providing the liquid supply port 58 at the protruding position, the liquid supply surface 74 to form the liquid supply port 58 may also be configured as a surface substantially parallel to the gas flow direction P1 and the axis P2. With this, a liquid atomized by the atomization portion M may smoothly be guided along the liquid supply surface 74.

As illustrated in FIG. 14 and FIG. 16, each of the first inclined surface 70, the second inclined surface 72, the liquid supply surface 74, and the third inclined surface 76 of Embodiment 1 has a curved surface shape. In Embodiment 1, in particular, the curved surface shape has an arc shape with a constant curvature.

As illustrated in FIG. 16, in a plan view direction of the gas supply port 52, the ridge lines 78, 80, and 82 each have a shape approaching an upstream side (arrow Q2) of the liquid supply port 58 in the liquid flow direction Q1 as the ridge lines 78, 80, and 82 each leave from the gas supply port 52 in the lateral direction (X-direction). Similarly, the first inclined surface 70, the second inclined surface 72, the liquid supply surface 74, and the third inclined surface 76 each have a shape approaching the upstream side of the liquid flow direction Q1.

When a gas blown out from the gas supply port 52 rises slightly spreading in the X-direction being the lateral direction, variation in the distances to the liquid supply port 58 is reduced. This is because the first inclined surface 70, the second inclined surface 72, and the ridge line 78 connecting the surfaces have a shape approaching the upstream side (arrow Q2). With this, the liquid discharged from the liquid supply port 58 is more uniformly merged with the gas flow P, thereby achieving uniform atomization. This leads to an increase in the atomization amount.

Further, in Embodiment 1, the first inclined surface 70, the second inclined surface 72, the liquid supply surface 74, and the third inclined surface 76 each have a smooth curved surface shape, and the ridge lines 78, 80, and 82 also have a gently curved shape in plan view. With this, turbulence is less likely to occur in the gas blown out from the gas supply port 52, the gas flow P smoothly rises, and a flow velocity may be maintained. This makes the Venturi effect be easily exhibited.

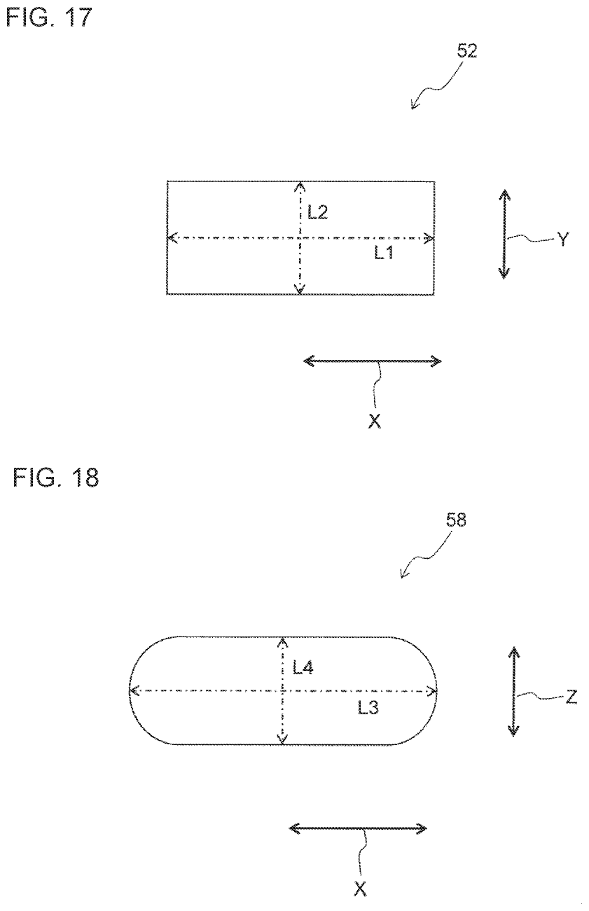

Next, FIG. 17 and FIG. 18 are plan views of the gas supply port 52 and the liquid supply port 58, respectively.

As illustrated in FIG. 17, the gas supply port 52 of Embodiment 1 forms a laterally-long rectangular opening. The gas supply port 52 has a lateral width L1 and a longitudinal width L2. The lateral width L1 is a length along the X-direction corresponding to a lateral direction of the gas supply port 52, and the longitudinal width L2 is a length along the Y-direction corresponding to a longitudinal direction of the gas supply port 52. The lateral width L1 is the maximum measurement of the gas supply port 52 in the lateral direction, and the longitudinal width L2 is the maximum measurement of the gas supply port 52 in the longitudinal direction. In Embodiment 1, the lateral width L1 is set to be larger than the longitudinal width L2.

By forming the gas supply port 52 in a laterally-long shape, the flow P of the gas blown out from the gas supply port 52 may be made to rise while spreading in the lateral direction. This allows the negative pressure to be generated in a wide range. With this, atomization may be performed in a wide range, and thus the atomization amount may be increased and a particle diameter may be made smaller.

As illustrated in FIG. 18, the liquid supply port 58 of Embodiment 1 forms a laterally-long opening in which a semicircle and a semicircle are connected by two straight lines. The liquid supply port 58 has a lateral width L3 and a longitudinal width L4. The lateral width L3 is a length along the X-direction corresponding to a lateral direction of the liquid supply port 58, and the longitudinal width L4 is a length along the Z-direction corresponding to a longitudinal direction of the liquid supply port 58. The lateral width L3 is the maximum measurement of the liquid supply port 58 in the lateral direction, and the longitudinal width L4 is the maximum measurement of the liquid supply port 58 in the longitudinal direction. In Embodiment 1, the lateral width L3 is set to be larger than the longitudinal width L4.

By forming the liquid supply port 58 in a laterally-long shape, the liquid supply port 58 may widely receive the negative pressure generated in a wide range in response to the flow P of the gas blown out while spreading in the lateral direction, and the range of the atomization may be widened. This leads to an increase in the atomization amount and a decrease in the particle diameter.

As described above, the atomizer 2 of Embodiment 1 is an atomizer for mixing a liquid and a gas to atomize, and includes the gas supply member 50 and the liquid supply member 56. The gas supply member 50 is provided with the gas flow path 54 and the gas supply port 52 configured to supply a gas, and the liquid supply member 56 is provided with the liquid flow path 66 and the liquid supply port 58 configured to supply a liquid. The gas supply member 50 has the gas supply surface 68 as a surface to form the gas supply port 52. The liquid supply port 58 is opened toward the axis P2 orthogonal to the gas supply surface 68 at the gas supply port 52. The liquid supply member 56 has the first inclined surface 70 between the liquid supply port 58 and the gas supply port 52. In a section including the gas flow path 54 and the liquid flow path 66 (the section illustrated in FIG. 15, also referred to as the “first section”), the first inclined surface 70 is inclined to leave from the axis P2 as the first inclined surface 70 extends away from the gas supply surface 68. The liquid supply port 58 is located at a position protruding from the virtual plane 84 including the first inclined surface 70.

With the configuration described above, by providing the first inclined surface 70, negative pressure may be generated in accordance with the flow P of a gas from the gas supply port 52, and a liquid may be sucked out from the liquid supply port 58 with the Venturi effect and atomized. Further, by providing the liquid supply port 58 at a position protruding from the virtual plane 84 including the first inclined surface 70, the negative pressure around the liquid supply port 58 becomes high, and a large amount of liquid may be sucked out from the liquid supply port 58. With this, the atomization amount may be increased.

Note that, with respect to the first inclined surface 70, restatement as follows may be possible. That is, as illustrated in FIG. 15, the liquid supply member 56 has a wall portion W restricting a space H, in which a gas discharged from the gas supply port 52 flows, in a direction (lateral direction) intersecting with the gas flow direction P1. The wall portion W is constituted by the first inclined surface 70, the second inclined surface 72, the liquid supply surface 74, and the third inclined surface 76. The wall portion W has the first inclined surface 70 on the upstream side relative to the liquid supply port 58 in the gas flow direction P1, and the first inclined surface 70 is inclined relative to the gas flow direction P1 such that the space H expands along the gas flow direction P1. Although the description above is omitted in Modification 1 to Modification 8 to be described later, the same restatement may be possible.

Further, in the atomizer 2 of Embodiment 1, the liquid supply member 56 has the second inclined surface 72 on the upstream side relative to the first inclined surface 70 in the gas flow direction P1 and at a position facing the flow P of the gas blown out from the gas supply port 52. In the section illustrated in FIG. 15, the second inclined surface 72 is inclined to approach the axis P2 as the second inclined surface 72 extends away from the gas supply surface 68. With the configuration described above, by providing the second inclined surface 72, the flow P of the gas supplied from the gas supply port 52 may be made to collide with the second inclined surface 72 to change the direction thereof. Further, as illustrated in FIG. 15, in a case that the second inclined surface 72 has such a shape as to cover the width of the gas supply port 52, the flow velocity of the air after the collision with the second inclined surface 72 increases.

In the atomizer 2 of Embodiment 1, the first inclined surface 70 and the second inclined surface 72 are connected to each other by the ridge line 78. With the configuration described above, by forming the first inclined surface 70 and the second inclined surface 72 continuously by the ridge line 78, the negative pressure generated around the first inclined surface 70 may be made higher, and the liquid supply port 58 may be also disposed at a position close to the negative pressure generation portion.

In the atomizer 2 of Embodiment 1, the ridge line 78 has a shape that approaches the upstream side (arrow Q2) of the liquid supply port 58 in the liquid flow direction Q1 as the ridge line 78 goes away from the gas supply port 52, in a plan view direction of the gas supply port 52 as illustrated in FIG. 16. With the configuration described above, variation in the distances from any position of the ridge line 78 to the liquid supply port 58 is reduced in comparison with a case that the shape of the ridge line 78 is a straight line. With this, variation in the negative pressure around the ridge line 78 is reduced, and the atomization may be performed in a wider range. This leads to an increase in the atomization amount and a decrease in the particle diameter.

In the atomizer 2 of Embodiment 1, the liquid supply member 56 further has the liquid supply surface 74 to form the liquid supply port 58. With the configuration described above, by providing the liquid supply surface 74, the liquid supply port 58 may easily be formed.

In the atomizer 2 according to Embodiment 1, the liquid supply surface 74 extends substantially parallel to the axis P2 at the gas supply port 52. With the configuration described above, the atomized droplets may be guided in a desired direction along the liquid supply surface 74.

In the atomizer 2 of Embodiment 1, the lateral width L1, being the maximum measurement of the opening of the gas supply port 52 in the lateral direction (X-direction) orthogonal to the first section illustrated in FIG. 15, is larger than the longitudinal width L2, being the maximum measurement of the gas supply port 52 in the longitudinal direction (Y-direction) intersecting with the lateral direction. With the configuration described above, the negative pressure may be generated in a wider range.

In the atomizer 2 of Embodiment 1, the lateral width L3, being the maximum measurement of the opening of the liquid supply port 58 in the lateral direction (X-direction) orthogonal to the first section illustrated in FIG. 15, is larger than the longitudinal width L4, being the maximum measurement of the opening of the liquid supply port 58 in the longitudinal direction (Z-direction) intersecting with the lateral direction. With the configuration described above, the negative pressure generated in a wide range may widely be received by the liquid supply port 58, and the atomization amount may be increased.

The atomizer 2 of Embodiment 1 further includes the piezoelectric pumps 26 and 28 configured to supply a gas to the gas supply port 52. With the configuration described above, when the piezoelectric pumps 26 and 28 having a smaller output than a motor pump or the like are used, the action of increasing the atomization amount may more effectively be achieved.

In the atomizer 2 of Embodiment 1, the gas supply member 50 and the liquid supply member 56 are separate members. With the configuration described above, the degree of freedom in designing each member is improved.

(Modification 1 to Modification 8)

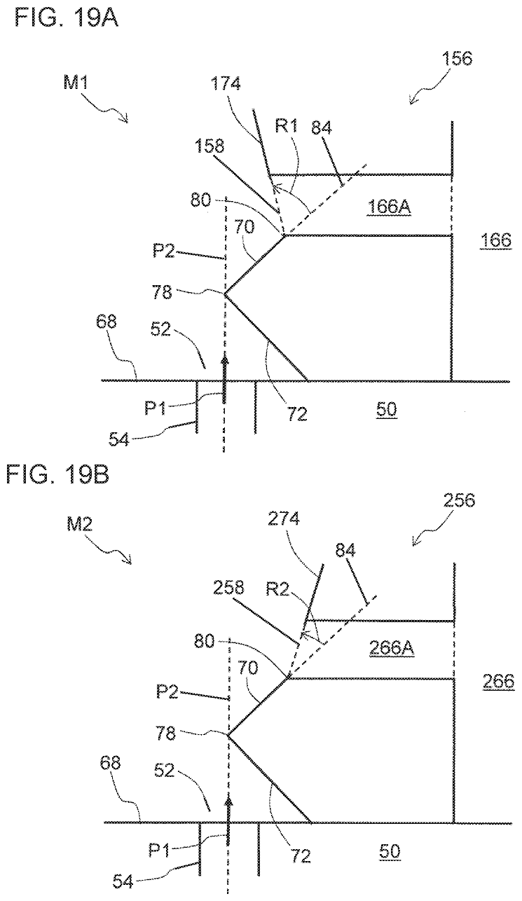

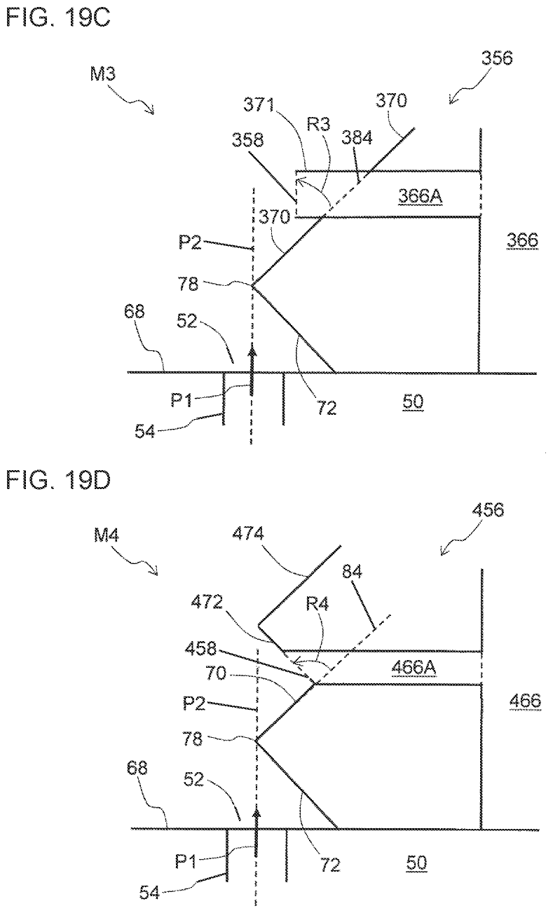

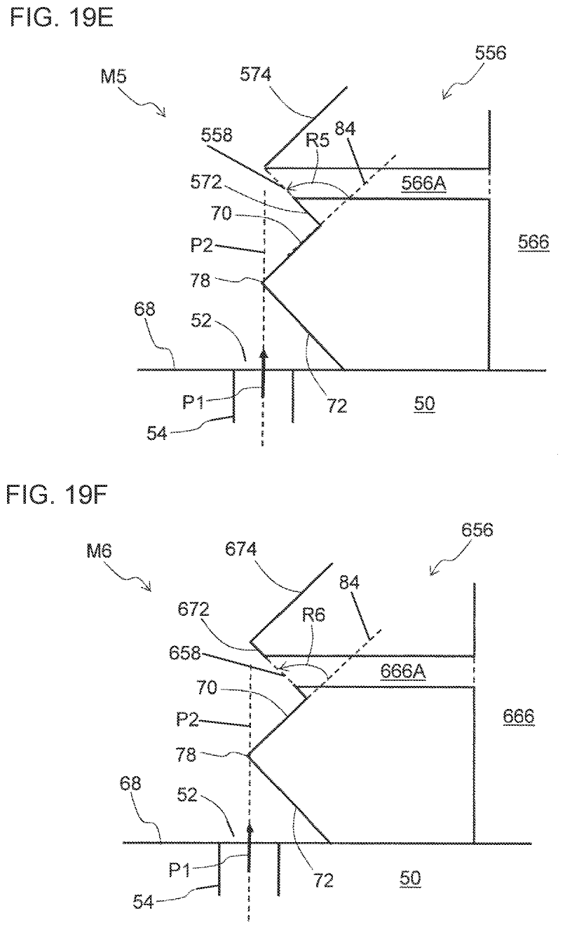

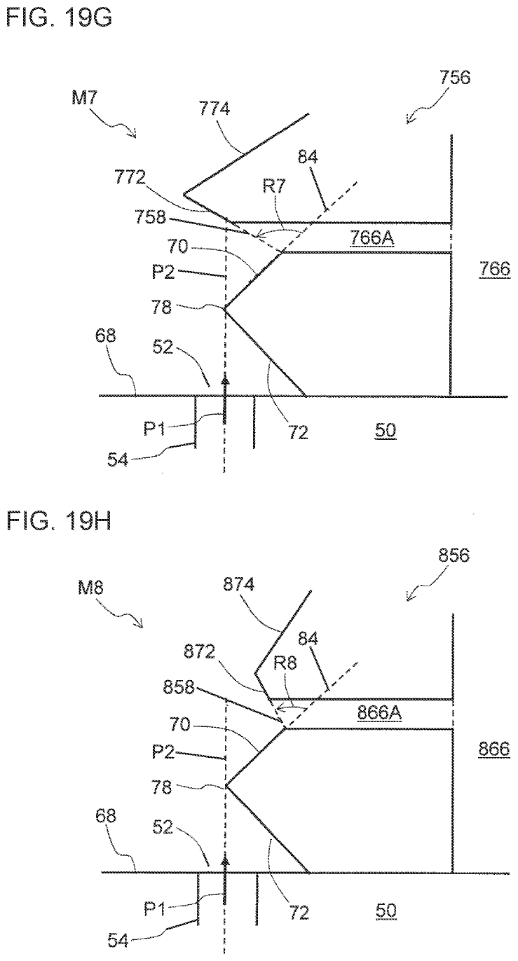

Next, modifications of the sectional shape of the liquid supply member 56 will be described with reference to FIG. 19A to FIG. 19H.

FIG. 19A is a longitudinal sectional view of an atomization portion M1 including a liquid supply member 156 according to Modification 1. Modification 1 is different from Embodiment 1 in that a liquid supply surface 174 to form a liquid supply port 158 is inclined relative to the gas flow direction P1 at the gas supply port 52.

In the example illustrated in FIG. 19A, the liquid supply surface 174 is inclined in a direction approaching the axis P2 including the gas flow direction P1 as the liquid supply surface 174 extends away from the gas supply surface 68. A shrunk diameter portion 166A of a liquid flow path 166 extends to the liquid supply port 158 formed in the liquid supply surface 174. With the configuration described above, the liquid supply port 158 is disposed at a position more protruding from the virtual plane 84 including the first inclined surface 70 than the liquid supply port 58 of Embodiment 1 (see arrow R1). With this, the negative pressure generated around the liquid supply port 158 may be increased, and the atomization amount may be increased. Note that the first inclined surface 70 is provided on the upstream side relative to the liquid supply port 158 in the gas flow direction P1 as part of a wall portion W1 restricting a space H1, through which a gas discharged from the gas supply port 52 flows, in a direction intersecting with the gas flow direction P1. The first inclined surface is inclined relative to the gas flow direction P1 to expand the space H1 along the gas flow direction P1.

FIG. 19B is a longitudinal sectional view of an atomization portion M2 including a liquid supply member 256 according to Modification 2. Modification 2 is different from Embodiment 1, in the same manner as in Modification 1, in that a liquid supply surface 274 to form a liquid supply port 258 is inclined relative to the gas flow direction P1.

In the example illustrated in FIG. 19B, the liquid supply surface 274 is inclined in a direction leaving from the axis P2 including the gas flow direction P1 as the liquid supply surface 274 extends away from the gas supply surface 68. A shrunk diameter portion 266A of a liquid flow path 266 extends to the liquid supply port 258 formed in the liquid supply surface 274. Even in the case above, the liquid supply port 258 is disposed at a position protruding from the virtual plane 84 including the first inclined surface 70 (see arrow R2). With this, in the same manner as in Embodiment 1 and Modification 1, by increasing the negative pressure generated around the liquid supply port 258, the effect of increasing the atomization amount may be achieved. Note that the first inclined surface 70 is provided on an upstream side relative to the liquid supply port 258 in the gas flow direction P1 as part of a wall portion W2 restricting a space H2, through which a gas discharged from the gas supply port 52 flows, in a direction intersecting with the gas flow direction P1. The first inclined surface 70 is inclined relative to the gas flow direction P1 to expand the space H2 along the gas flow direction P1.

FIG. 19C is a longitudinal sectional view of an atomization portion M3 including a liquid supply member 356 according to Modification 3. Modification 3 is different from Embodiment 1 in that a liquid supply port 358 is provided at a locally protruding position on a first inclined surface 370.

In the example illustrated in FIG. 19C, the first inclined surface 370 has a protrusion portion 371. The protrusion portion 371 is a portion obtained by extending a shrunk diameter portion 366A of a liquid flow path 366, and has a cylindrical shape, for example. Even in the case above, the liquid supply port 358 is disposed at a position protruding from a virtual plane 384 including the first inclined surface 370 (see arrow R3). With this, in the same manner as in Embodiment 1 and other modifications, by increasing the negative pressure generated around the liquid supply port 358, the effect of increasing the atomization amount may be achieved.

FIG. 19D is a longitudinal sectional view of an atomization portion M4 including a liquid supply member 456 according to Modification 4. Modification 4 is different from Embodiment 1 in that a liquid supply surface 472 to form a liquid supply port 458 is an inclined surface.

In the example illustrated in FIG. 19D, the liquid supply surface 472 is inclined in a direction approaching the axis P2 including the gas flow direction P1 as the liquid supply surface 472 extends away from the gas supply surface 68. A shrunk diameter portion 466A of a liquid flow path 466 extends to the liquid supply port 458 formed in the liquid supply surface 472. Even in the case above, the liquid supply port 458 is disposed at a position protruding from the virtual plane 84 including the first inclined surface 70 (see arrow R4), and the effect of increasing the atomization amount may be achieved.

FIG. 19E is a longitudinal sectional view of an atomization portion M5 including a liquid supply member 556 according to Modification 5. Modification 5 is different from Modification 4 illustrated in FIG. 19D in that a liquid supply port 558 formed in a liquid supply surface 572 is provided at a position adjacent to a third inclined surface 574. A shrunk diameter portion 566A of a liquid flow path 566 extends to the liquid supply port 558 formed in the liquid supply surface 572. Even in the case above, the liquid supply port 558 is disposed at a position protruding from the virtual plane 84 including the first inclined surface 70 (arrow R5), and the effect of increasing the atomization amount may be achieved.

FIG. 19F is a longitudinal sectional view of an atomization portion M6 including a liquid supply member 656 according to Modification 6. Modification 6 is different from Modification 4 and Modification 5 in that a liquid supply port 658 formed in a liquid supply surface 672 is provided at an intermediate position adjacent to neither the first inclined surface 70 nor a third inclined surface 674. A shrunk diameter portion 666A of a liquid flow path 666 extends to the liquid supply port 658 formed in the liquid supply surface 672. Even in the case above, the liquid supply port 658 is disposed at a position protruding from the virtual plane 84 including the first inclined surface 70 (arrow R6), and the effect of increasing the atomization amount may be achieved.

FIG. 19G is a longitudinal sectional view of an atomization portion M7 including a liquid supply member 756 according to Modification 7. Modification 7 is different from Modification 4 illustrated in FIG. 19D in that a liquid supply surface 772 and a third inclined surface 774 to form a liquid supply port 758 are inclined to protrude more than the first inclined surface 70 and the second inclined surface 72. A shrunk diameter portion 766A of a liquid flow path 766 extends to the liquid supply port 758 formed in the liquid supply surface 772. Even in the case above, the liquid supply port 758 is disposed at a position protruding from the virtual plane 84 including the first inclined surface 70 (arrow R7), and the atomization amount may be increased.

FIG. 19H is a longitudinal sectional view of an atomization portion M8 including a liquid supply member 856 according to Modification 8. Modification 8 is different from the Modification 7 illustrated in FIG. 19G in that the first inclined surface 70 and the second inclined surface 72 are inclined to protrude more than a liquid supply surface 872 and a third inclined surface 874 to form a liquid supply port 858. A shrunk diameter portion 866A of a liquid flow path 866 extends to the liquid supply port 858 formed in the liquid supply surface 872. Even in the case above, the liquid supply port 858 is disposed at a position protruding from the virtual plane 84 including the first inclined surface 70 (arrow R8), and the effect of increasing the atomization amount may be achieved.

### Embodiment 2

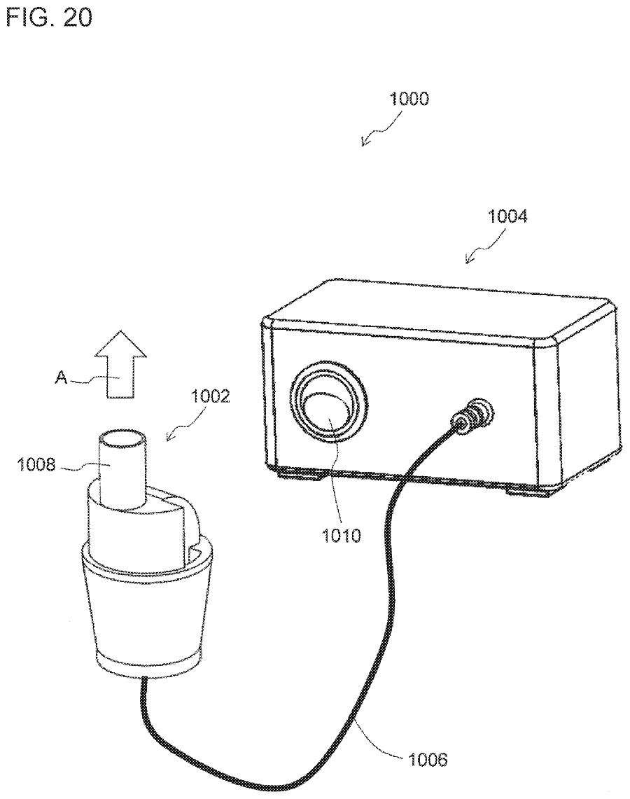

An atomizer according to Embodiment 2 of the present disclosure will be described with reference to FIG. 20. In Embodiment 2, differences from Embodiment 1 will mainly be described. The same or equivalent components are denoted by the same reference signs, and a description thereof will be omitted.

An atomizer 1002 of Embodiment 2 is different from the atomizer 2 of Embodiment 1 in being used as part of a nonportable nebulizer device 1000 instead of a handheld nebulizer.

FIG. 20 is a perspective view of the nebulizer device 1000 including the atomizer 1002 according to Embodiment 2.

The nebulizer device 1000 illustrated in FIG. 20 includes the atomizer 1002, a case 1004, and a tube 1006.

The atomizer 1002 is a member corresponding to the first case 10 and the second case 12 in the atomizer 2 of Embodiment 1. The atomizer 1002 incorporates an atomization portion M (not illustrated) similar to the atomizer 2 of Embodiment 1, and the compressed air supplied from the case 1004 and a liquid are mixed to atomize. The atomized liquid is blown out from a blowout nozzle 1008 (see arrow A).

The case 1004 is a unit configured to supply the compressed air to the atomizer 1002. The case 1004 corresponds to the third case 14 in the atomizer 2 of Embodiment 1, and incorporates components (not illustrated) such as a piezoelectric pump and a board for generating the compressed air. A switch 1010 for driving is provided on a front surface of the case 1004. When a user presses the switch 1010, the compressed air is generated inside the case 1004 and is supplied to the atomizer 1002 through the tube 1006.

Since the internal structure of the atomizer 1002 is the same as the internal structure of the first case 10 and the second case 12 in the atomizer 2 of Embodiment 1, a description thereof will be omitted.

With the use of the nonportable nebulizer device 1000 as illustrated in FIG. 20, the user can hold the atomizer 1002 connected to the case 1004 and make use of the atomized liquid be blown out from the blowout nozzle 1008. Further, since the atomizer 1002 of Embodiment 2 includes the atomization portion M having the same structure as that of the atomizer 2 of Embodiment 1, the effect of increasing the atomization amount may similarly be achieved.

Although the present disclosure has been described with reference to Embodiment 1 and Embodiment 2 described above, the present disclosure is not limited thereto. For example, although the two piezoelectric pumps 26 and 28 are provided in the embodiment described above, the present disclosure is not limited to that case, and one or three or more piezoelectric pumps may be provided.

Although the present disclosure has been fully described in connection with preferred embodiments with reference to the accompanying drawings, various changes and modifications will become apparent to those skilled in the art. It is to be understood that such variations and modifications are included in the scope of the present disclosure according to the appended claims as long as not departing therefrom. Further, a change in the combination and the order of elements in each embodiment can be realized without departing from the scope and spirit of the present disclosure.

The present disclosure is useful for an atomizer for medical use, cosmetic use, or the like.
2 ATOMIZER
4 CASE
6 BLOWOUT NOZZLE
8 SWITCH
10 FIRST CASE
12 SECOND CASE
14 THIRD CASE
16 MARK
17 POWER SUPPLY COVER
18 BOTTOM SURFACE
19 SUPPORT MEMBER
20 POWER SUPPLY PLUG PORTION
22, 24 CONTROL BOARD
26 PIEZOELECTRIC PUMP
26A UPSTREAM END
26B DOWNSTREAM END
28 PIEZOELECTRIC PUMP
28A UPSTREAM END
28B DOWNSTREAM END
32, 34, 36, 38, 39 MOUNT PORTION
40 NOZZLE PORTION
40A UPSTREAM END
40B DOWNSTREAM END
41 UPPER SURFACE PORTION
42, 44 CONNECTION FLOW PATH MEMBER
46 OPENING
50 GAS SUPPLY MEMBER
52 GAS SUPPLY PORT
54 GAS FLOW PATH
54A SHRUNK DIAMETER PORTION
55 LIQUID RESERVOIR
55A BOTTOM SURFACE
55B INNER PERIPHERAL SURFACE
56 LIQUID SUPPLY MEMBER
58 LIQUID SUPPLY PORT
59 LIQUID SUCTION PORT
60 MOUNT PORTION
62 FLOW PATH FORMING PORTION
64 UPPER END PORTION
66 LIQUID FLOW PATH
66A SHRUNK DIAMETER PORTION
68 GAS SUPPLY SURFACE
70 FIRST INCLINED SURFACE
72 SECOND INCLINED SURFACE
74 LIQUID SUPPLY SURFACE
76 THIRD INCLINED SURFACE
78, 80, 82 RIDGE LINE
84 VIRTUAL PLANE
156 LIQUID SUPPLY MEMBER
158 LIQUID SUPPLY PORT
166 LIQUID FLOW PATH
166A SHRUNK DIAMETER PORTION
174 LIQUID SUPPLY SURFACE
256 LIQUID SUPPLY MEMBER
258 LIQUID SUPPLY PORT
266 LIQUID FLOW PATH
266A SHRUNK DIAMETER PORTION
274 LIQUID SUPPLY SURFACE
356 LIQUID SUPPLY MEMBER
358 LIQUID SUPPLY PORT
366 LIQUID FLOW PATH
366A SHRUNK DIAMETER PORTION
370 FIRST INCLINED SURFACE
371 PROTRUSION PORTION
384 VIRTUAL PLANE
456 LIQUID SUPPLY MEMBER
458 LIQUID SUPPLY PORT
466 LIQUID FLOW PATH
466A SHRUNK DIAMETER PORTION
472 LIQUID SUPPLY SURFACE
474 THIRD INCLINED SURFACE
556 LIQUID SUPPLY MEMBER
558 LIQUID SUPPLY PORT
566 LIQUID FLOW PATH
566A SHRUNK DIAMETER PORTION
572 LIQUID SUPPLY SURFACE
574 THIRD INCLINED SURFACE
656 LIQUID SUPPLY MEMBER
658 LIQUID SUPPLY PORT
666 LIQUID FLOW PATH
666A SHRUNK DIAMETER PORTION
672 LIQUID SUPPLY SURFACE
674 THIRD INCLINED SURFACE
756 LIQUID SUPPLY MEMBER
758 LIQUID SUPPLY PORT
766 LIQUID FLOW PATH
766A SHRUNK DIAMETER PORTION
772 LIQUID SUPPLY SURFACE
774 THIRD INCLINED SURFACE
856 LIQUID SUPPLY MEMBER
858 LIQUID SUPPLY PORT
866 LIQUID FLOW PATH
866A SHRUNK DIAMETER PORTION
872 LIQUID SUPPLY SURFACE
874 THIRD INCLINED SURFACE
1000 NEBULIZER DEVICE
1002 ATOMIZER
1004 CASE
1006 TUBE
1008 BLOWOUT NOZZLE
1010 SWITCH

1. An atomizer for mixing a gas and a liquid to atomize, the atomizer comprising:
a gas supply member having a gas flow path and a gas supply port configured to supply a gas; and
a liquid supply member having a liquid flow path and a liquid supply port configured to supply a liquid,
wherein the gas supply member has a gas supply surface that forms the gas supply port,
wherein the liquid supply port is open toward an axis orthogonal to the gas supply surface at the gas supply port,
wherein the liquid supply member has a first inclined surface between the liquid supply port and the gas supply port,
wherein the first inclined surface is inclined to leave from the axis as the first inclined surface extends away from the gas supply surface in a first section, the first section including the gas flow path and the liquid flow path, and
wherein the liquid supply port protrudes from a plane including the first inclined surface.

2. The atomizer according to claim 1,
wherein the liquid supply member has a second inclined surface on an upstream side of the first inclined surface in a gas flow direction, and faces a gas blown out from the gas supply port, and
wherein in the first section, the second inclined surface is inclined to approach the axis as the second inclined surface extends away from the gas supply surface.

3. The atomizer according to claim 2, wherein the first inclined surface and the second inclined surface are connected by a ridge line.

4. The atomizer according to claim 3, wherein the ridge line has a shape approaching an upstream side of the liquid supply port in a liquid flow direction as the ridge line leaves from the gas supply port in a plan view direction of the gas supply port.

5. The atomizer according to claim 1, wherein the liquid supply member further has a liquid supply surface that forms the liquid supply port.

6. The atomizer according to claim 5, wherein the liquid supply surface extends substantially parallel to the axis at the gas supply port.

7. The atomizer according to claim 1, wherein a maximum measurement of an opening of the liquid supply port in a lateral direction orthogonal to the first section is larger than a maximum measurement of the opening of the liquid supply port in a longitudinal direction intersecting with the lateral direction.

8. The atomizer according to claim 1, wherein a maximum measurement of the gas supply port in a lateral direction orthogonal to the first section is larger than a maximum measurement of the gas supply port in a longitudinal direction intersecting with the lateral direction.

9. The atomizer according to claim 1, further comprising a piezoelectric pump configured to supply the gas to the gas supply port.

10. The atomizer according to claim 1, wherein the gas supply member and the liquid supply member are separate members.
